(19) United States
(12) Patent Application Publication
(10) Pub. No.: US 2010/0174292 A1
(43) Pub. Date: Jul. 8, 2010

(54) Surgical instrument for placing a prosthesis into a target area of a living subject

(75) Inventors: Robert F. Labadie, Nashville, TN (US); Jason E. Mitchell, Greenbrier, TN (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: VANDERBILT UNIVERSITY, Nashville, TN (US)

(21) Appl. No.: 12/683,800

(22) Filed: Jan. 7, 2010

Related U.S. Application Data
(60) Provisional application No. 61/143,076, filed on Jan. 7, 2009.
(60) Provisional application No. 61/183,722, filed on Jun. 3, 2009.

(51) Int. Cl. A61F 11/00

(52) U.S. Cl. 606/108

(57) ABSTRACT

One aspect of the present invention relates to a surgical instrument for placing a prosthesis into a target area of a living subject. In one embodiment, the surgical instrument includes a body member, a shaft extending away from the body member for securing the prosthesis, an engaging member engaged with the body member and the shaft, for engaging the prosthesis, and an actuator connected to the engaging member, for moving between a first position in which the engaging member is disengaged from the prosthesis and a second position in which the engaging member is engaged with the prosthesis.

### CROSS-REFERENCE TO RELATED PATENT APPLICATION

This application claims priority to and the benefit of, pursuant to 35 U.S.C. §119(e), U.S. provisional patent application Ser. No. 61/143,076, filed Jan. 7, 2009, entitled “SURGICAL INSTRUMENT FOR PLACING A PROSTHESIS INTO A TARGET AREA OF A LIVING SUBJECT,” by Robert F. Labadie, and U.S. provisional patent application Ser. No. 61/183,722, filed Jun. 3, 2009, entitled “SURGICAL INSTRUMENT FOR PLACING A PROSTHESIS INTO A TARGET AREA OF A LIVING SUBJECT,” by Robert F. Labadie, et al., the contents of which are incorporated herein in its entireties by reference.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference were individually incorporated by reference.

### FIELD OF THE INVENTION

The present invention generally relates to a surgical instrument, and in particular to a surgical instrument for placing a stapes prosthesis into an inner ear of a living subject.

### BACKGROUND OF THE INVENTION

The placement of a stapes prosthesis is used to improve hearing in a living subject, and the prosthesis can be placed into a target area of the inner ear of the living subject through a surgical procedure performed by a medical professional. Conventional instruments used for placement of the prosthesis include alligator clips, pliers, or micrograppers. However, these instruments suffer from several drawbacks. One such drawback is that conventional instruments do not allow the medical professional performing the procedure to place the prosthesis at a user-defined angle, i.e., they cannot be customized to the angle of placement according to the shape of a particular patient's ear, for example. In addition, conventional instruments for placement of a stapes prosthesis do not allow the medical professional to place the prosthesis into the target area using only one action, or one touch. For instance, more than one finger or hand may be required to safely and effectively place the prosthesis into the target area. Another drawback is that conventional means provide only one plane of support to secure the prosthesis, such as from beneath the prosthesis or touching one side of it, and thus the prosthesis is not held as securely in place as it could be if it was secured by forces acting on it in more than one direction.

Therefore, a heretofore unaddressed need exists in the art to address the aforementioned deficiencies and inadequacies.

### SUMMARY OF THE INVENTION

The present invention, in one aspect, relates to a surgical instrument for placing a prosthesis into a target area of a living subject. The target area is an inner ear of a living subject. In one embodiment, the surgical instrument includes a body member having a first end portion, an opposite, second end portion, and a body portion defined therebetween having a longitudinal axis, wherein the body portion defines a bore therein along the longitudinal axis; a bendable shaft extending away from the first end portion of the body member, wherein the bendable shaft has a first end portion configured to secure the prosthesis, an opposite, second end portion connected to the first end portion of the body member, and a body portion defined therebetween and forming a bore therein in communication with the bore of the body member; an engaging member received in the bores of the body member and the shaft, wherein the engaging member has a first end configured to engage or disengage with the prosthesis and disposed proximate the first end portion of the shaft, and an opposite, second end secured within the bore of the body portion; and an actuator slidably engaged with the body portion of the body member and connected to the second end of the engaging member, adapted for moving along the body member between a first position in which the first end of the engaging member is disengaged from the prosthesis and a second position in which the first end of the engaging member is engaged with the prosthesis.

In operation, the prosthesis engaged with the one or more grippers is guided to the target area of the living subject by the body member and the shaft, the actuator is moved from the second position to the first position such that the engaging member is urged through the bores of the body member and the shaft to disengage the prosthesis secured at the first end portion of the shaft to allow the prosthesis to be placed into the target area of the living subject.

In one embodiment, the engaging member includes one or more wires. The prosthesis includes a body portion and a hook portion extending therefrom, wherein the first end portion of the bendable shaft is configured to secure at least one of the body portion and the hook portion.

In another aspect, the present invention relates to a surgical instrument for placing a prosthesis into a target area of a living subject. In one embodiment, the surgical instrument has a body member having a first end portion, an opposite, second end portion, and a body portion defined therebetween having a longitudinal axis, wherein the body portion defines a bore therein along the longitudinal axis; a shaft extending away from the first end of the body member, wherein the shaft has a first end portion configured to secure the prosthesis, an opposite, second end portion connected to the first end portion of the body member, and a body portion defined therebetween and forming a bore therein in communication with the bore of the body member; an engaging member received in the bore of the body member and the bore of the shaft, being movable therein, wherein the engaging member has a first end configured to engage or disengage with the prosthesis and disposed proximate the first end of the shaft, and an opposite, second end secured within the bore of the body portion; and an actuator slidably engaged with the body portion of the body member and connected to the second end of the engaging member.

In one embodiment, the shaft is bendable. The engaging member includes one or more wires.

In one embodiment, the actuator is adapted for moving between a first position in which the first end of the engaging member is disengaged from the prosthesis and a second position in which the first end of the engaging member is engaged with the prosthesis, respectively.

In one embodiment, the prosthesis includes a body portion and a hook portion extending therefrom, wherein the first end portion of the shaft is configured to secure at least one of the body portion and the hook portion, wherein the first end of the engaging member is configured to engage or disengage with the body portion of the prosthesis.

For such a surgical instrument in operation, the prosthesis engaged with the one or more grippers is guided to the target area of the living subject by the body member and the shaft, the actuator is moved from the second position to the first position such that the engaging member is urged through the bores of the body member and the shaft to disengage the prosthesis secured at the first end portion of the shaft to allow the prosthesis to be placed into the target area of the living subject.

In yet another aspect, the present invention relates to a surgical instrument for placing a prosthesis into a target area of a living subject. In one embodiment, the surgical instrument has a body member; a shaft extending away from the body member for securing the prosthesis; an engaging member engaged with the body member and the shaft, for engaging the prosthesis; and an actuator connected to the engaging member, for moving between a first position in which the engaging member is disengaged from the prosthesis and a second position in which the engaging member is engaged with the prosthesis.

In one embodiment, the actuator is slidably engaged with the body member. In operation, the prosthesis engaged with the one or more grippers is guided to the target area of the living subject by the body member and the shaft, the actuator is moved from the second position to the first position such that the engaging member is urged through the bores of the body member and the shaft to disengage the prosthesis secured at the first end portion of the shaft to allow the prosthesis to be placed into the target area of the living subject.

In a further aspect, the present invention relates to a surgical instrument for placing a prosthesis into a target area of a living subject. In one embodiment, the surgical instrument has a body member having a first end portion, an opposite, second end portion, a body portion defined therebetween having a longitudinal axis, and a tab extending from the first end portion along a direction that defines an angle relative to the longitudinal axis, wherein the body portion defines a passage therein along the longitudinal axis, and the tab is adapted for cradling or supporting the prosthesis during a surgical procedure; one or more grippers received in the passage of the body member, having a working end portion configured to engage or disengage with the prosthesis and disposed proximate the first end portion of the body member; and an actuator engaged with the body member and the one or more grippers, being movable along the body member between a first position in which the one or more grippers are disengaged from the prosthesis and a second position in which the one or more grippers are engaged with the prosthesis.

In operation, the prosthesis engaged with the one or more grippers is guided to the target area of the living subject by the body member, the actuator is moved from the second position to the first position such that the one or more grippers are urged through the body member to disengage the prosthesis from the working end portion of the one or more grippers so as to be place the prosthesis into the target area of the living subject.

In one embodiment, the angle is less than 180° and greater than 0. Preferably, the angle is about 90°.

In one embodiment, the passage is in the form of at least one of a bore and a slot.

In one embodiment, the actuator comprises a sliding button defining a hole, and wherein when the actuator is engaged with the body member, the body portion of the body member is received in the hole.

In one embodiment, the one or more grippers are configured such that when the one or more grippers are extended, the one or more grippers move away from the center axis of the body member, while the one or more grippers move toward the center axis of the body member when the one or more grippers are retracted. In one embodiment, the one or more grippers comprise one or more wires. Further, the working end portion of the one or more grippers may have a claw-like member.

In one embodiment, the prosthesis is preloaded in the surgical instrument in a manufacturer.

The surgical instrument may be disposable or reusable.

In yet a further aspect, the present invention relates to a surgical instrument for placing a prosthesis into a target area of a living subject. In one embodiment, the surgical instrument has a body member having a first end portion, an opposite, second end portion, and a body portion defined therebetween having a longitudinal axis, wherein the body portion defines a passage therein along the longitudinal axis; one or more grippers received in the passage of the body member, having a working end portion configured to engage or disengage with the prosthesis and disposed proximate the first end portion of the body member; and an actuator engaged with the body member and the one or more grippers, being movable along the body member between a first position in which the one or more grippers are disengaged from the prosthesis and a second position in which the one or more grippers are engaged with the prosthesis.

In one embodiment, the body member further has a tab extending from the first end portion along a direction that defines an angle relative to the longitudinal axis, and wherein the angle is less than 180° and greater than 0. Preferably, the angle is about 90°. The tab is adapted for cradling or supporting the prosthesis during a surgical procedure.

In one embodiment, the passage is in the form of at least one of a bore and a slot.

In one embodiment, the actuator comprises a sliding button defining a hole, and wherein when the actuator is engaged with the body member, the body portion of the body member is received in the hole.

In one embodiment, the one or more grippers are configured such that when the one or more grippers are extended, the one or more grippers move away from the center axis of the body member, while the one or more grippers move toward the center axis of the body member when the one or more grippers are retracted. The one or more grippers may include one or more wires. The working end portion of the one or more grippers may have a claw-like member.

These and other aspects of the present invention will become apparent from the following description of the preferred embodiments, taken in conjunction with the following drawings, although variations and modifications therein may be affected without departing from the spirit and scope of the novel concepts of the disclosure.

### BRIEF DESCRIPTION OF THE DRAWINGS

FIG. 1 shows schematically a perspective view of a surgical instrument according to one embodiment of the present invention, where FIGS. 1A and 1B show partially enlarged cross-sectional views.

FIG. 2A shows schematically a partially perspective view of the surgical instrument shown in FIG. 1, where an actuator is shown in a first position; FIG. 2B shows schematically a partially perspective view of the surgical instrument shown in FIG. 1, where the actuator is shown in a second position.

FIG. 3A shows schematically a partially perspective view of the first end of a shaft and an engaging member of the surgical instrument, according to one embodiment of the present invention; FIG. 3B shows schematically a partially perspective view of the first end of the shaft shown in the embodiment of FIG. 3A, securing a prosthesis.

FIG. 4 shows schematically an area of the inner ear of a living subject that includes a target area for receiving the prosthesis, as placed using a surgical instrument according to one embodiment of the present invention.

FIG. 5 shows schematically a perspective view of a surgical instrument according to another embodiment of the present invention, where FIG. 5A shows a partially enlarged view.

FIG. 6 shows schematically another perspective view of the surgical instrument shown in FIG. 5, where FIG. 6A shows a partially enlarged view.

FIG. 7 shows schematically an exploding view of the surgical instrument shown in FIG. 5, where FIGS. 7A and 7B show two partially enlarged views.

### DETAILED DESCRIPTION OF THE INVENTION

The present disclosure is more particularly described in the following examples that are intended as illustrative only since numerous modifications and variations therein will be apparent to those skilled in the art. Various embodiments of the disclosure are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. The use of examples anywhere in this specification, including examples of any terms discussed herein, is illustrative only, and in no way limits the scope and meaning of the disclosure or of any exemplified term. Likewise, the disclosure is not limited to various embodiments given in this specification.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the terms “comprising,” “including,” “having,” “containing,” “involving,” and the like are to be understood to be open-ended, i.e., to mean including but not limited to.

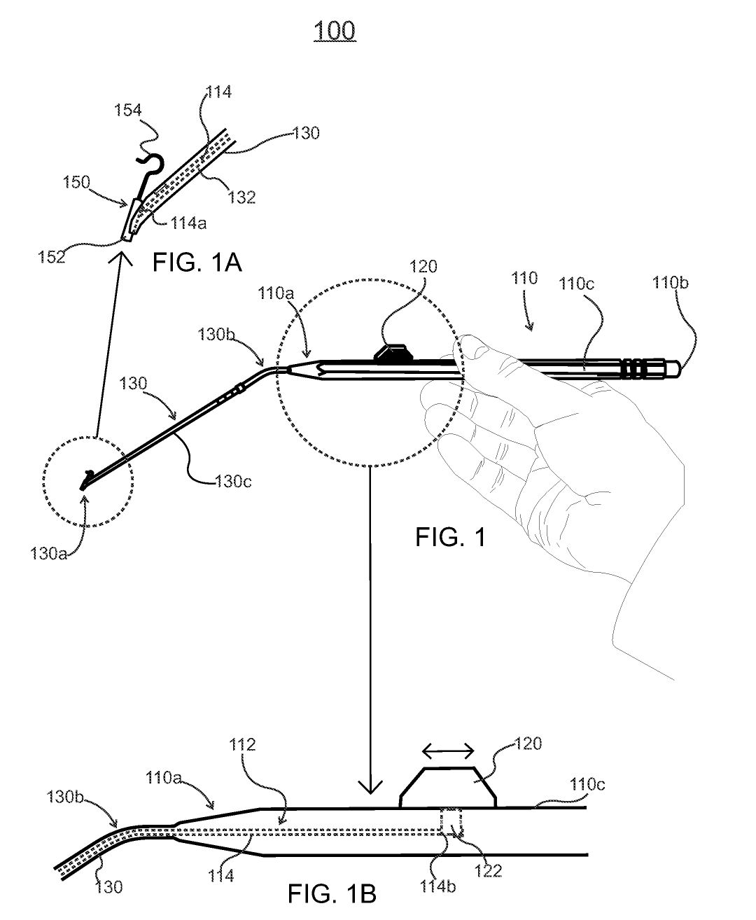

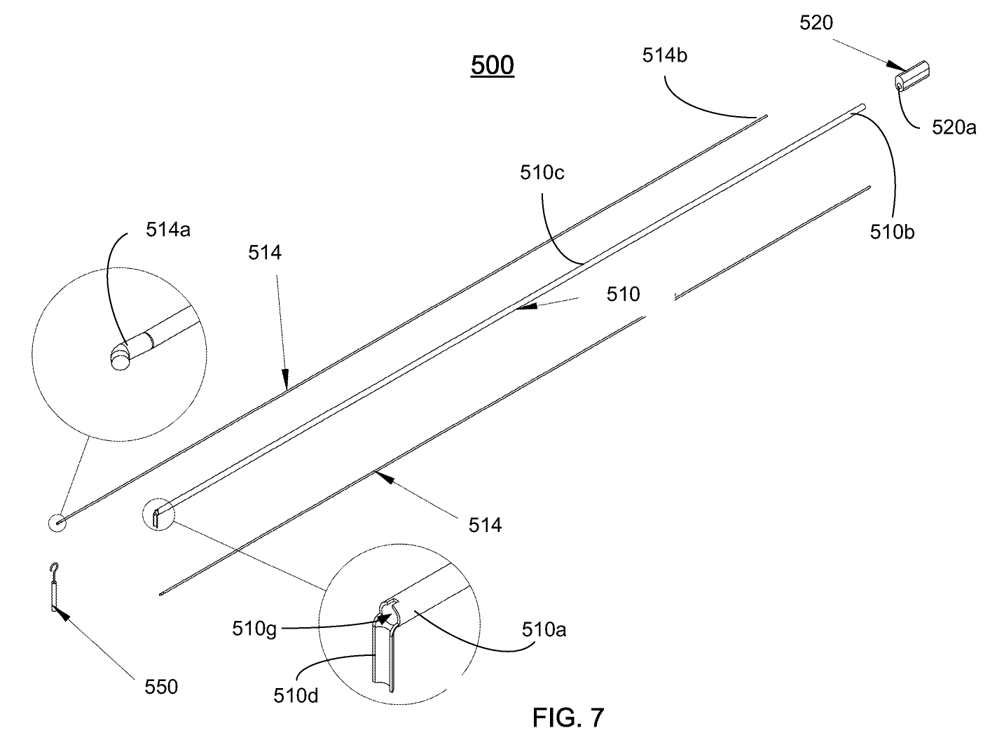

The description will be made as to the embodiments of the present invention in conjunction with the accompanying drawings in FIGS. 1-7. In accordance with the purposes of this invention, as embodied and broadly described herein, this invention, in one aspect, relates to a surgical instrument for placing a prosthesis into a target area of a living subject.

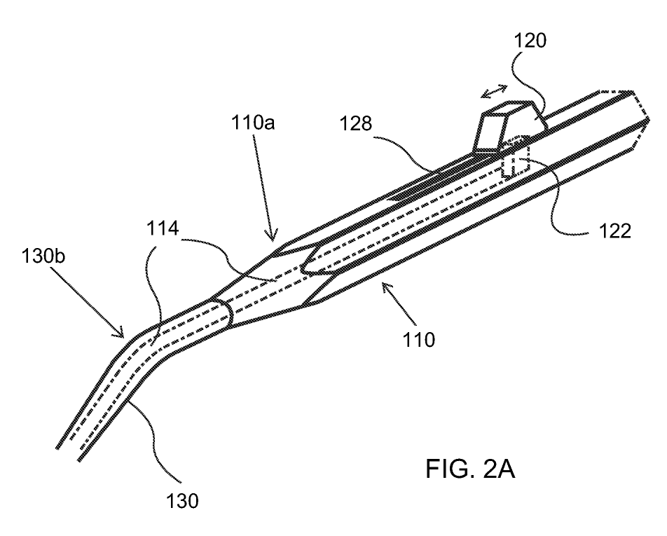

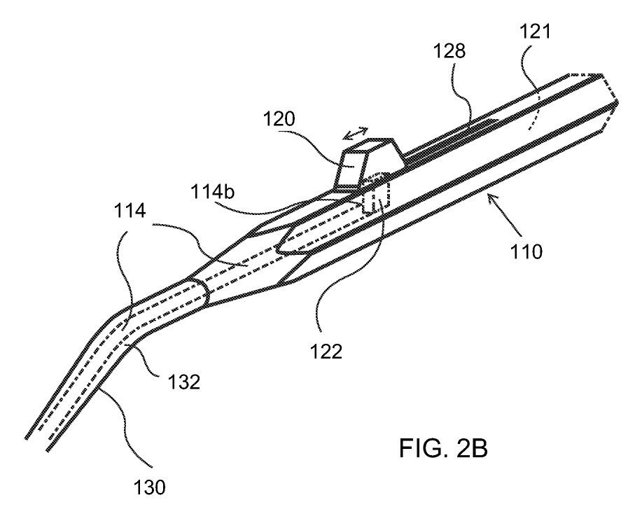

Now referring to FIGS. 1-4, a surgical instrument 100 for placing a prosthesis 150 into a target area 409 of a living subject is shown. In this exemplary embodiment, the surgical instrument 100 includes a body member 110, a shaft 130, an engaging member 114 and an actuator 120. The body member 110 has a first end portion 110a, an opposite, second end portion 110b, and a body portion 110c defined between the first and second end portions 110a and 110b. The body portion 110c having a longitudinal axis forms a bore 112 inside along the longitudinal axis. As shown in FIG. 1, the shaft 130 extends away from the first end 110a of the body member 110. The shaft 130 has a first end portion 130a configured to secure the prosthesis 150, an opposite, second end portion 130b connected to the first end portion 110a of the body member 110, and a body portion 130c defined between the first and second end portions 130a and 130b and forming a bore 132 therein, which is in communication with the bore 112 of the body member 110. The engaging member 114 has a first end 114a configured to engage or disengage with the prosthesis 150, disposed proximate the first end 130a of the shaft 130, and an opposite, second end 114b secured within the bore 112 of the body portion 110. The engaging member 114 is received in the bore 112 of the body member 110 and the bore 132 of the shaft 130 and is movable therein. The actuator 120 is slidably engaged with the body portion 110c of the body member 110 and is connected to the second end 114b of the engaging member 114. As shown in the enlarged, cross-sectional partial view of FIG. 1B, the actuator 120 is connected to the second end 114b of the engaging member 114 through a protruding member 122 inside the bore 112 of the body member 110. Also as shown in FIG. 1, the body member 110 and the shaft 130 have substantially circular cross-sections (see also FIGS. 2A and 2B). It would be appreciated for people skilled in the art that the body member 110 and the shaft 130 may be formed to have other shapes of cross-sections.

The shaft 130 may be bendable or flexible. This allows the user, for example a medical professional, to place the prosthesis 150 at a user-defined angle to allow for an adjustable angle of placement according to the shape of the target area 409. Further, the engaging member 114 includes one or more wires. The actuator 120 is adapted for moving along the body portion 110c of the body member 110 between a first position in which the first end 114a of the engaging member 114 is disengaged from the prosthesis 150 (see FIG. 2B) and a second position in which the first end 114a of the engaging member 114 is engaged with the prosthesis 150, respectively (see FIG. 2A).

For such a surgical instrument 100 in operation, the prosthesis 150 engaged with the surgical instrument 100 is guided to the target area 409, e.g., an inner ear of a patient, then, the actuator 120 is moved from the second position to the first position so that the engaging member 114 is urged through the bore 112 of the body member 110 and the bore 132 of the shaft 130 to disengage with the prosthesis 150 secured at the first end 130a of the shaft 130, hereby allowing the prosthesis 150 to be placed into the target area of the living subject. This permits the user to deploy and place the prosthesis into the target area 409 using only one action, or one touch, i.e., only one finger on one hand is required to place the prosthesis 150 into the target area 409.

As shown in the enlarged partial view shown in FIG. 1A, the prosthesis 150 in one embodiment includes a body portion 152 and a hook portion 154 extending from the body portion 152, where the first end portion 130a of the shaft 130 is configured to secure at least one of the body portion 152 and the hook portion 154. The first end 114a of the engaging member 114 is configured to engage the body portion 152 of the prosthesis 150. The first end 114a can alternatively be configured to engage the hook portion 152.

FIG. 2A shows schematically a partially perspective view of the surgical instrument 100 shown in FIG. 1, where the actuator 220 is shown in a first position in which the first end 114a of the engaging member 114 is disengaged from the prosthesis 150.

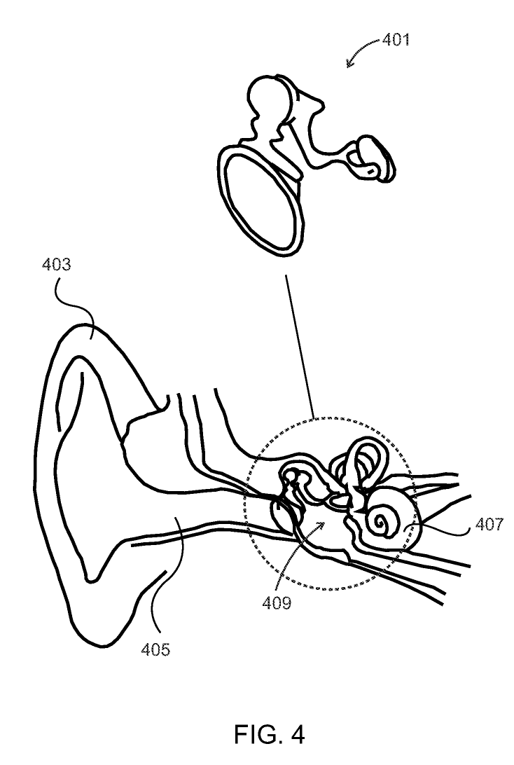

FIG. 2B shows schematically a partially perspective view of the surgical instrument of FIG. 1A, where the actuator 120 is in a second position in which the engaging member 114 is engaged with the prosthesis 150. As shown, the actuator 120 is slidably engaged with the body member 110 and moves within slot 128 between the first and second position, which translates the movement to urge the engaging member 114, through the connection of the second end 114b of the engaging member 114 at the protruding member 122 of the actuator 120. The engaging member 114, in turn, urges the prosthesis (i.e. deploys it) into place in the target area (409, FIG. 4). The engaging member 114 can then be disengaged (i.e. released) from the prosthesis when the actuator 120 is moved back from the second position to the first position.

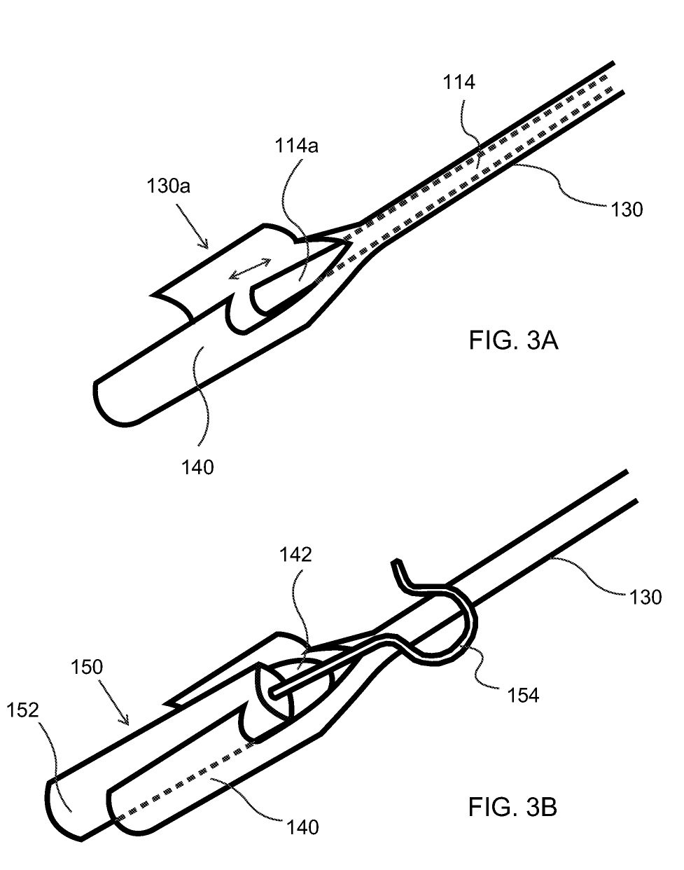

FIG. 3A shows schematically a partially perspective view of the first end 130a of the shaft 130 and the first end 114a of the engaging member 114 of the surgical instrument 100 shown in FIG. 1. As shown, the first end 130a of the shaft 130 has a semi-cylindrical scoop-shaped portion. As shown in FIG. 3B, this scoop portion conforms to the shape of the outer surface of the body portion 152 (connected to the hook portion 154) of the prosthesis 150, to secure it in place as the surgical instrument 100 is moving the prosthesis 150 into a target area (see FIG. 4) to be placed. In this way, more than one plane of support can apply to secure the prosthesis 150, and can be securely held in place by forces acting in more than one direction. An adhesive or water, represented at 142, can also be used to provide surface tension to supplement the security for the prosthesis 150.

FIG. 4 shows schematically an area of the ear of a living subject, which includes the outer ear 403, ear canal 405, and middle ear cavity 409, where the middle ear cavity 409 is a target area for placement of a prosthesis (not shown) that can replace the stapes, shown in the enlarged view as 401, where the prosthesis is placed using a surgical instrument according to one embodiment of the present invention.

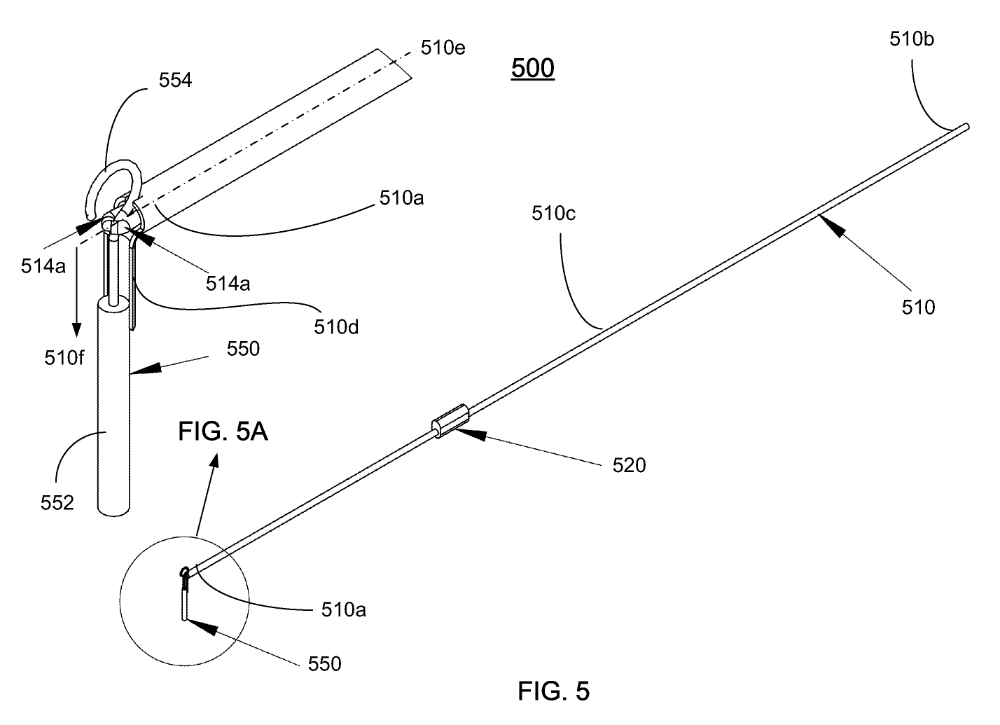

Referring to FIGS. 5-7, a surgical instrument 500 is shown according to another embodiment of the present invention. In this embodiment, the surgical instrument 500 has a body member 510 having a first end portion 510a, an opposite, second end portion 510b, a body portion 510c defined therebetween having a longitudinal axis 510e, and a tab 510d extending from the first end portion 510a along a direction 510f that defines an angle relative to the longitudinal axis 510e. The body portion 510c defines a passage 510g therein along the longitudinal axis 510e. The passage 510g is in the form of at least one of a bore and a slot. In the exemplary embodiment, the passage 510g includes a bore. The tab 510d is adapted for cradling or supporting the prosthesis during a surgical procedure. The angle is less than 180° and greater than 0. Preferably, the angle is about 90°.

The surgical instrument 500 also includes one or more grippers 514 received in the passage 510g of the body member 510, having a working end portion 414a configured to engage or disengage with the prosthesis and disposed proximate the first end portion 510a of the body member 510. The one or more grippers 514 are configured such that when the one or more grippers 514 are extended, the one or more grippers 514 move away from the center axis 510e of the body member 510, while the one or more grippers 514 move toward the center axis 510e of the body member 510 when the one or more grippers 514 are refracted. The one or more grippers 514 comprise one or more wires. Further, the working end portion 514a of the one or more grippers 514 may have a claw-like member. The current design relates to a stapes-seat or a surgical instrument which may be called as the “claw” embodiment as it has one or more opposable grippers that holds the prosthetic in a way that resembles the claw of a bird.

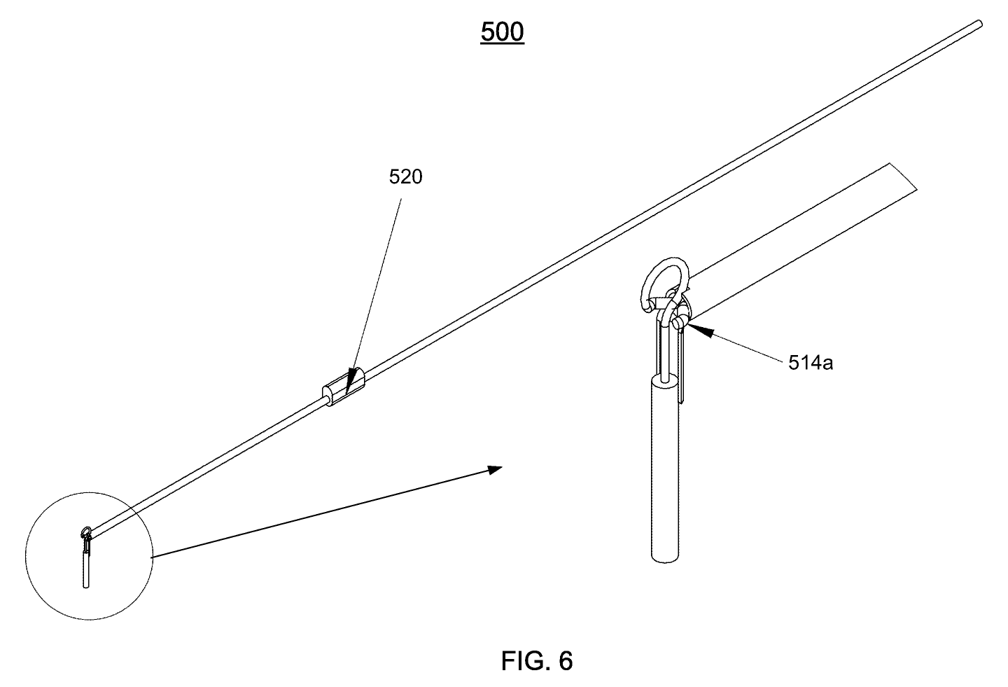

The surgical instrument 500 further includes an actuator 520 engaged with the body member 510 and the one or more grippers 514, being movable along the body member 510 between a first position in which the one or more grippers 514 are disengaged from the prosthesis 550, as shown in FIG. 6A, and a second position in which the one or more grippers 514 are engaged with the prosthesis 550, as shown in FIG. 5A. The actuator may comprise a sliding button defining a hole 520a. As assembled, the actuator 520 is engaged with the body member 510, the body portion 510c of the body member 510 is received in the hole 520a.

For such a surgical instrument 500 in operation, the prosthesis 520 engaged with the one or more grippers 514 is guided to the target area of the living subject by the body member 510, the actuator 520 is moved from the second position to the first position such that the one or more grippers 514 are urged through the body member 510 to disengage the prosthesis 550 from the working end portion 514a of the one or more grippers 514 so as to be place the prosthesis 550 into the target area of the living subject.

In one embodiment, the prosthesis is preloaded in the surgical instrument in a manufacturer. The surgical instrument may be disposable or reusable.

Alternatively, the body member may include a tube. The center of the tube is hollow allowing for the passage of the one of more grippers. Along the body of the tube is a hole or slot which allows access to the one of more grippers. Access to the one of more grippers may also be accomplished via the other end of the body member. The sliding button has a feature such as a hole that allows it to be attached slidably to the body member and another feature which allows it to be attached to the one of more grippers. The one of more grippers comprises a pre formed wire with features in one end which are shaped to grip the stapes prosthetic. Other features or bends are shaped in such a way that the one of more grippers moves away from the center axis of the body member when it is extended and toward the axis when retracted. There may be another feature which allows the one of more grippers to be joined to the sliding button.

In operation, the sliding button is pushed forward so that the one of more grippers are moved away from the axis to the open position. A stapes prosthetic would be placed onto the end of the body member and the sliding button would be pulled backward closing the one of more grippers around the stapes prosthetic.

The stapes prosthetic is then placed within the middle ear using the body member as a support and guide structure. Next the sliding button is pushed forward releasing the prosthetic.

In one embodiment, the prosthesis may be preloaded—that is comes form the manufacturer with the surgical instrument and prosthesis as one assembly. Accordingly, the present invention provides a preloaded insertion tool for any middle ear implant (this may include other ossicular chain prostheses such as partial ossicular replacement prostheses (PORPs) and total ossicular replacement prostheses (TORPs).

Thus the surgical instrument of manufacture according to the present invention has two forms: (a) a stand alone device used by the surgeon to place a prosthesis; and/or (b) a device and prosthesis combination that comes preloaded from the manufacturer where the device may become disposable after the procedure.

Embodiments of the present invention generally relate to a novel stapes-seat that can accurately and safely place a stapes prosthetic into the middle ear of a human patient.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

What is claimed is:

1. A surgical instrument for placing a prosthesis into a target area of a living subject, comprising:
(a) a body member having a first end portion, an opposite, second end portion, and a body portion defined therebetween having a longitudinal axis, wherein the body portion defines a bore therein along the longitudinal axis;
(b) a bendable shaft extending away from the first end of the body member and substantially coaxial with the body member, wherein the bendable shaft has a first end portion configured to secure the prosthesis, an opposite, second end portion connected to the first end portion of the body member, and a body portion defined therebetween and forming a bore therein in communication with the bore of the body member;
(c) an engaging member received in the bores of the body member and the shaft, wherein the engaging member has a first end configured to engage or disengage with the prosthesis and disposed proximate the first end portion of the shaft, and an opposite, second end secured within the bore of the body portion; and
(d) an actuator slidably engaged with the body portion of the body member and connected to the second end of the engaging member, adapted for moving along the body member between a first position in which the first end of the engaging member is disengaged from the prosthesis and a second position in which the first end of the engaging member is engaged with the prosthesis.

2. The surgical instrument of claim 1, wherein in operation, the prosthesis engaged with the one or more grippers is guided to the target area of the living subject by the body member and the shaft, the actuator is moved from the second position to the first position such that the engaging member is urged through the bores of the body member and the shaft to disengage the prosthesis secured at the first end portion of the shaft to allow the prosthesis to be placed into the target area of the living subject.

3. The surgical instrument of claim 1, wherein the engaging member includes one or more wires.

4. The surgical instrument of claim 1, wherein the prosthesis includes a body portion and a hook portion extending therefrom, wherein the first end portion of the bendable shaft is configured to secure at least one of the body portion and the hook portion.

5. The surgical instrument of claim 1, wherein the target area is an inner ear of a living subject.

6. A surgical instrument for placing a prosthesis into a target area of a living subject, comprising:
(a) a body member having a first end portion, an opposite, second end portion, and a body portion defined therebetween having a longitudinal axis, wherein the body portion defines a bore therein along the longitudinal axis;
(b) a shaft extending away from the first end of the body member, wherein the shaft has a first end portion configured to secure the prosthesis, an opposite, second end portion connected to the first end portion of the body member, and a body portion defined therebetween and forming a bore therein in communication with the bore of the body member;
(c) an engaging member received in the bore of the body member and the bore of the shaft, being movable therein, wherein the engaging member has a first end configured to engage or disengage with the prosthesis and disposed proximate the first end of the shaft, and an opposite, second end secured within the bore of the body portion; and
(d) an actuator slidably engaged with the body portion of the body member and connected to the second end of the engaging member.

7. The surgical instrument of claim 6, wherein the shaft is bendable.

8. The surgical instrument of claim 6, wherein the engaging member includes one or more wires.

9. The surgical instrument of claim 6, wherein the actuator is adapted for moving between a first position in which the first end of the engaging member is disengaged from the prosthesis and a second position in which the first end of the engaging member is engaged with the prosthesis, respectively.

10. The surgical instrument of claim 9, wherein in operation, the prosthesis engaged with the one or more grippers is guided to the target area of the living subject by the body member and the shaft, the actuator is moved from the second position to the first position such that the engaging member is urged through the bores of the body member and the shaft to disengage the prosthesis secured at the first end portion of the shaft to allow the prosthesis to be placed into the target area of the living subject.

11. The surgical instrument of claim 6, wherein the prosthesis includes a body portion and a hook portion extending therefrom, wherein the first end portion of the shaft is configured to secure at least one of the body portion and the hook portion.

12. The surgical instrument of claim 11, wherein the first end of the engaging member is configured to engage or disengage with the body portion of the prosthesis.

13. A surgical instrument for placing a prosthesis into a target area of a living subject, comprising:
(a) a body member;
(b) a shaft extending away from the body member for securing the prosthesis;
(c) an engaging member engaged with the body member and the shaft, for engaging the prosthesis; and
(d) an actuator connected to the engaging member, for moving between a first position in which the engaging member is disengaged from the prosthesis and a second position in which the engaging member is engaged with the prosthesis.

14. The surgical instrument of claim 13, wherein the actuator is slidably engaged with the body member.

15. The surgical instrument of claim 13, wherein in operation, the prosthesis engaged with the one or more grippers is guided to the target area of the living subject by the body member and the shaft, the actuator is moved from the second position to the first position such that the engaging member is urged through the bores of the body member and the shaft to disengage the prosthesis secured at the first end portion of the shaft to allow the prosthesis to be placed into the target area of the living subject.

16. A surgical instrument for placing a prosthesis into a target area of a living subject, comprising:
(a) a body member having a first end portion, an opposite, second end portion, a body portion defined therebetween having a longitudinal axis, and a tab extending from the first end portion along a direction that defines an angle relative to the longitudinal axis, wherein the body portion defines a passage therein along the longitudinal axis, and the tab is adapted for cradling or supporting the prosthesis during a surgical procedure;
(b) one or more grippers received in the passage of the body member, having a working end portion configured to engage or disengage with the prosthesis and disposed proximate the first end portion of the body member; and
(c) an actuator engaged with the body member and the one or more grippers, being movable along the body member between a first position in which the one or more grippers are disengaged from the prosthesis and a second position in which the one or more grippers are engaged with the prosthesis.

17. The surgical instrument of claim 16, wherein in operation, the prosthesis engaged with the one or more grippers is guided to the target area of the living subject by the body member, the actuator is moved from the second position to the first position such that the one or more grippers are urged through the body member to disengage the prosthesis from the working end portion of the one or more grippers so as to be place the prosthesis into the target area of the living subject.

18. The surgical instrument of claim 16, wherein the angle is less than 180° and greater than 0.

19. The surgical instrument of claim 18, wherein the angle is about 90°.

20. The surgical instrument of claim 16, wherein the passage is in the form of at least one of a bore and a slot.

21. The surgical instrument of claim 16, wherein the actuator comprises a sliding button defining a hole, and wherein when the actuator is engaged with the body member, the body portion of the body member is received in the hole.

22. The surgical instrument of claim 16, wherein the one or more grippers are configured such that when the one or more grippers are extended, the one or more grippers move away from the center axis of the body member, while the one or more grippers move toward the center axis of the body member when the one or more grippers are retracted.

23. The surgical instrument of claim 12, wherein the one or more grippers comprise one or more wires.

24. The surgical instrument of claim 23, wherein the working end portion of the one or more grippers comprises a claw-like member.

25. The surgical instrument of claim 16, wherein the prosthesis is preloaded in the surgical instrument in a manufacturer.

26. The surgical instrument of claim 16, being disposable or reusable.

27. A surgical instrument for placing a prosthesis into a target area of a living subject, comprising:
(d) a body member having a first end portion, an opposite, second end portion, and a body portion defined therebetween having a longitudinal axis, wherein the body portion defines a passage therein along the longitudinal axis;
(e) one or more grippers received in the passage of the body member, having a working end portion configured to engage or disengage with the prosthesis and disposed proximate the first end portion of the body member; and
(f) an actuator engaged with the body member and the one or more grippers, being movable along the body member between a first position in which the one or more grippers are disengaged from the prosthesis and a second position in which the one or more grippers are engaged with the prosthesis.

28. The surgical instrument of claim 27, wherein the body member further has a tab extending from the first end portion along a direction that defines an angle relative to the longitudinal axis, and wherein the angle is less than 180° and greater than 0.

29. The surgical instrument of claim 28, wherein the angle is about 90°.

30. The surgical instrument of claim 28, wherein the tab is adapted for cradling or supporting the prosthesis during a surgical procedure.

31. The surgical instrument of claim 28, wherein the passage is in the form of at least one of a bore and a slot.

32. The surgical instrument of claim 28, wherein the actuator comprises a sliding button defining a hole, and wherein when the actuator is engaged with the body member, the body portion of the body member is received in the hole.

33. The surgical instrument of claim 28, wherein the one or more grippers are configured such that when the one or more grippers are extended, the one or more grippers move away from the center axis of the body member, while the one or more grippers move toward the center axis of the body member when the one or more grippers are retracted.

34. The surgical instrument of claim 33, wherein the one or more grippers comprise one or more wires.

35. The surgical instrument of claim 34, wherein the working end portion of the one or more grippers comprises a claw-like member.
